(19) United States
(12) Patent Application Publication
(10) Pub. No.: US 2010/0131019 A1
(43) Pub. Date: May 27, 2010

(54) FIXING ELEMENT FOR A BONE FRAGMENT

(76) Inventor: Günter Lob, Munich (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/312,405

(22) PCT Filed: Nov. 15, 2007
(86) PCT No.: PCT/EP2007/062410

§ 371 Date: Jan. 26, 2010

(30) Foreign Application Priority Data
Nov. 15, 2006 (DE) 102006054533.8

(51) Int. Cl. A61B 17/84

(52) U.S. Cl. 606/327

(57) ABSTRACT

A fixing element, in particular a peg, for fixing bone fragments, ligaments or the like, has at least two outer sub-elements which divide the fixing element in the longitudinal direction with respect to a separating surface between the at least two outer sub-elements. The at least two outer sub-elements are of such a configuration and are so arranged relative to each other that the fixing element is adapted to be transferred from a compact condition into a laterally or radially expanded condition. The fixing element is of a dovetail-like configuration at its proximal end for coupling to an instrument for holding and introducing the fixing element. The fixing element has two notches which extend on opposite sides of the fixing element transversely with respect to the separating surface between the at least two sub-elements and which respectively extend over the sub-elements and which are so shaped that the depth of the respective notch decreases towards the proximal end of the fixing element and thus the width of the fixing element increases from a viewing direction along the two notches towards the proximal end of the fixing element. The width of the fixing element at the proximal end thereof being smaller than in a longitudinal portion adjoining the notches in the distal direction.

### BACKGROUND OF THE INVENTION

1. Technical Field

The invention concerns an implantable fixing element, in particular a peg, for fixing for example bone fragments, that is to say a fixing element for surgical use.

2. Description of the Background Art

The fixing element is divided in the longitudinal direction into at least two outer sub-elements and can have an expansion element. Such fixing elements are of such a configuration that they are to be transferred from a compact condition into a (radially or laterally) expanded condition by means of the expansion element or a suitable tool.

Fixing elements of that kind as are known for example from WO 96/16607 can be used in the manner of a peg for fixing ligaments to a bone or for fixing bone fragments. For that purpose, the fixing element is fitted into a bore in the bone and then expanded by means of the expansion element. By virtue of the expansion effect, the fixing element is fixed to the two bone fragments and in that way also fixes the two bone fragments relative to each other.

In order to be able to firmly hold the two outer sub-elements by means of a suitable instrument at their respective proximal ends and to guide them to the desired location, various coupling structures are known, which make it possible to couple the fixing element to the instrument. The connection between the fixing element and the insertion instrument, that is to be produced by means of the respective coupling structure, must be on the one hand reliable so that the fixing element can be certain to be guided to its position of implantation, but then it must also be reliable in terms of separation in order to be able to reliably release the fixing element.

### SUMMARY OF THE INVENTION

The object of the invention is to provide a fixing element of the kind known from WO 96/16607, which permits reliable coupling to an instrument for insertion of the fixing element.

According to the invention that object is attained by a fixing element of the kind set forth in the opening part of this specification, which is of a dovetail-like configuration at its proximal end for coupling to an instrument for holding and introducing the fixing element and has two notches which extend on opposite sides of the fixing element transversely with respect to a separating surface between the sub-elements and which respectively extend over the sub-elements and which are so shaped that the depth of the respective notch decreases towards the proximal end of the fixing element and thus the width of the fixing element increases from a viewing direction along the two notches towards the proximal end of the fixing element, the width of the fixing element at the proximal end thereof being smaller than in a longitudinal portion adjoining the notches in the distal direction.

The notches are preferably respectively defined by two boundary surfaces, namely a distal boundary surface extending perpendicularly to the longitudinal axis of the fixing element and a proximal boundary surface extending at an acute angle towards the longitudinal axis of the fixing element. The notches are therefore of a kind of triangular cross-section respectively formed by two outside surfaces of the fixing element, which extend inclinedly towards each other. The distal boundary surface preferably extends perpendicularly to the longitudinal axis of the fixing element while the proximal boundary surface preferably extends at an angle of 60°±5° relative to the longitudinal axis of the fixing element.

In addition, in its compact condition, the fixing element is preferably of a cylindrical basic shape over the major part of its length and can therefore be optimally inserted into a bone in round bores.

Preferably, the width of the fixing element at its proximal end, by virtue of the two notches, is smaller than the diameter of the cylindrical basic shape by a dimension which corresponds somewhat more than the depth of a notch in the radial direction. In other words: each notch, in relation to the longitudinal portion of the fixing element, that is disposed distally of the notch, is just twice as deep as in relation to the proximal end of the fixing element.

A preferred fixing element is one which in its compact condition is of a cylindrical basic shape of a diameter of between 3 and 7 mm and a length of over 60 mm. In the longitudinal direction of the fixing element, longitudinal portions of round cross-section and longitudinal portions of stellate cross-section alternate. The stellate cross-section of the corresponding longitudinal portions is formed by prismatic longitudinal ribs on the outside of those longitudinal portions. The diameter of the longitudinal portions of round cross-section is smaller than the diameter of the longitudinal portions of stellate cross-section.

Inserting the expansion element which is of an oversize in relation to the cavity enclosed by the sub-elements means that the two sub-elements are driven apart in parallel relationship by a distance exactly predetermined by the oversize of the expansion element.

Such a fixing element can advantageously be introduced in its compact condition into a round bore which extends from the outside of the bone through the fracture surface into a further bone portion. When the fixing element according to the invention is introduced into such a bore from the outside it can then be expanded by means of the expansion element and in that way fixes the two bone portions together across the fracture surface. In that respect, it is provided that the fixing element is of an overlength and thus projects proximally out of the bore. The projecting portion of the fixing element can then be cut off.

Advantageously, the fixing element comprises a thermoplastic material so that the fixing element can be cut off by means of a hot wire. In the best-case scenario that additionally entails welding between the outer sub-elements of the fixing element and the expansion element.

Equally, it is advantageous if the material making up the elements of the fixing element is bioresorbable so that the fixing element is gradually absorbed by the body after healing of the fracture. A suitable biocompatible material is for example a polylactide.

The fixing element can in addition advantageously contain osteosynthesis-stimulating substances such as for example bone morphogenic proteins which further promote bone growth. Ideally, the material of the fixing element is so optimized that the delivery of such bone growth-promoting substances is effected in a suitable dose for the desired healing process.

In regard to the external geometry of the fixing element, it is desirable if the longitudinal portions of round cross-section are of a length of between 0.5 and 1.5 mm while the longitudinal portions of stellate cross-section are preferably of a length of between 1 and 3 mm. That geometry permits effective clawing engagement of the expanded fixing element in the respective bone.

In regard to the internal geometry of the fixing element, it is desirable if both the cavity and also the expansion element are of a substantially rectangular cross-section. In that case, the substantially rectangular cross-section of the expansion element is preferably rounded off at the corners. In the longitudinal direction, the expansion element distally has a longitudinal portion which narrows towards the distal end and which allows the expansion element to be introduced with that longitudinal portion leading into the cavity between the two outer sub-elements of the fixing element when the latter is in its compact condition.

It is further advantageous if the separating surface between the two outer sub-elements projects in such a way that even in the expanded condition of the fixing element the two sub-elements are at least not displaceable relative to each other in the longitudinal direction and if possible also not in the lateral direction. For that purpose one of the two sub-elements preferably has projections which protrude beyond a main separating surface and which engage into corresponding recesses in the respective other sub-element. Such projections are preferably associated with each third longitudinal portion of stellate cross-section.

### BRIEF DESCRIPTION OF THE DRAWINGS

The invention will now be described in greater detail by means of an embodiment by way of example with reference to the Figures in which:

FIG. 1 is an exploded perspective view of the fixing element with its two outer sub-elements and the expansion element,

FIG. 2 is a perspective view of the fixing element in its compact condition,

FIG. 3 shows various detail views of the fixing element of FIGS. 1 and 2,

FIG. 4 is a perspective view of the fixing element in its expanded condition,

FIG. 5 is a perspective view of an alternative fixing element in its compact condition,

FIG. 6 shows various detail views of the alternative fixing element of FIG. 5, and

FIG. 7 is a perspective view of the alternative fixing element in its expanded condition.

### DETAILED DESCRIPTION OF THE INVENTION

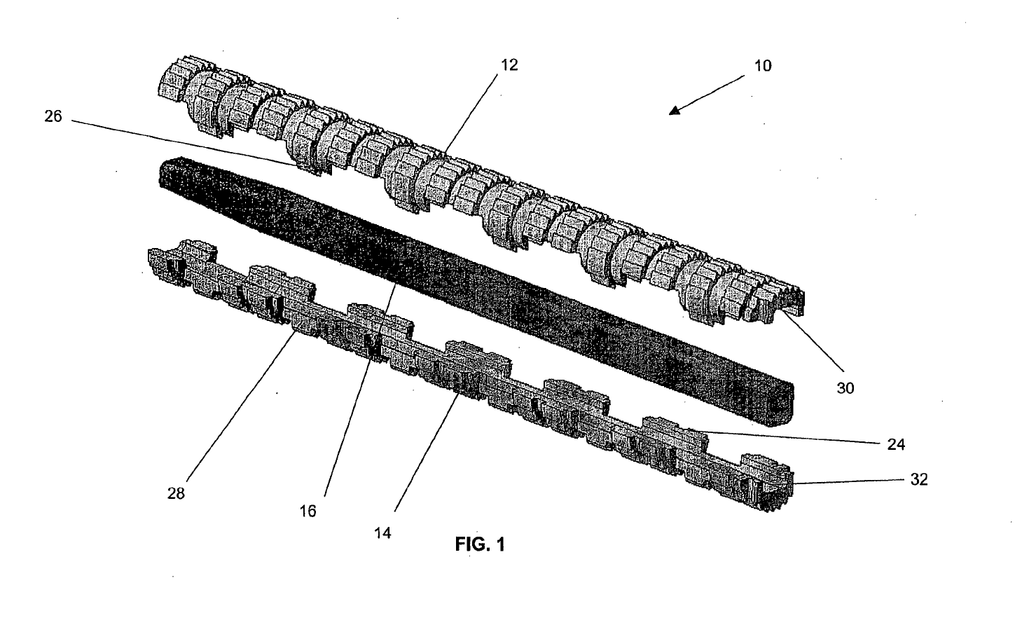

Referring to FIG. 1, therein illustrated is the preferred embodiment of a fixing element 10 made in accordance with the invention. This fixing element 10 has two outer sub-elements 12 and 14 which enclose an expansion element 16.

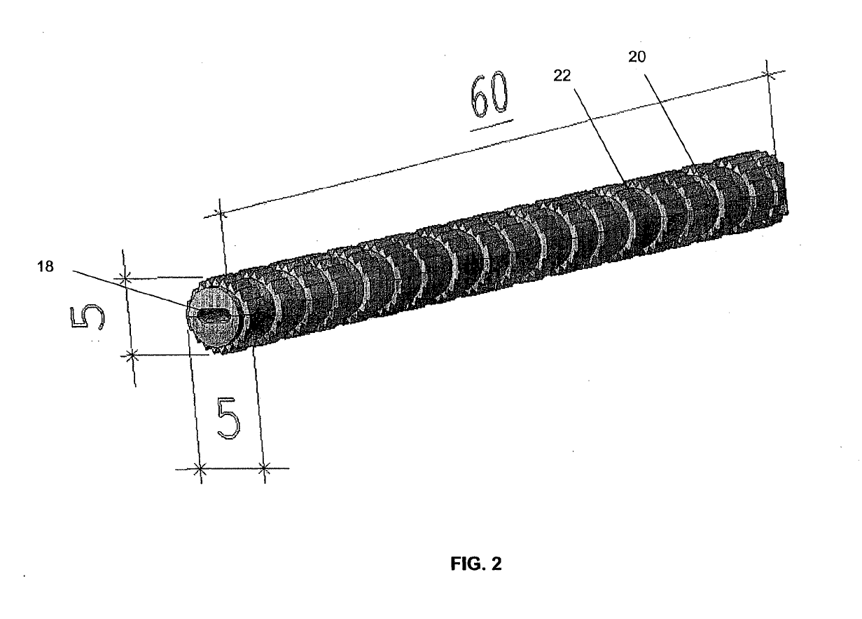

In FIG. 2, the fixing element 10 is shown in its compact condition in which the two outer sub-elements 12 and 14 bear directly against each other. It will be seen that the two sub-elements 12, 14 define a cavity 18 into which the expansion element 16 can be introduced.

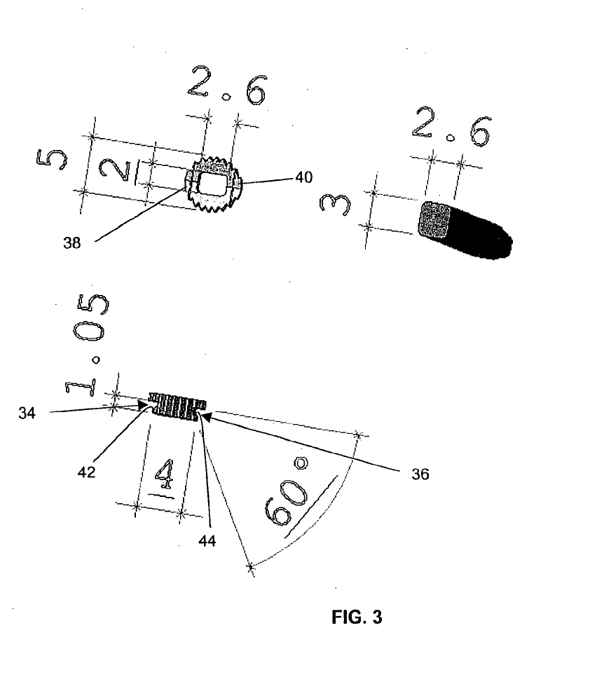

The top left view in FIG. 3 shows the proximal end of the fixing element 10 in its compressed condition, as is also shown in FIG. 2. Shown at bottom left in FIG. 3 is a plan view on to a short longitudinal portion of the fixing element 10 at the proximal end thereof. It is possible to clearly see from that plan view, two notches 34 and 36 into which an instrument for introducing the fixing element 10 can engage so that such an instrument is to be coupled to the fixing element 10 by way of the notches 34 and 36. The two notches 34 and 36 are respectively defined by a distal boundary surface 38 and 40 respectively and a proximal boundary surface 42 and 44 respectively. In that arrangement, the distal boundary surfaces 38 and 40 are in a plane extending perpendicularly to the longitudinal axis of the fixing element 10. The two proximal boundary surfaces 42 and 44 respectively extend at an angle of 60° towards the longitudinal axis of the fixing element 10. The width of the fixing element 10 in the viewing direction shown at bottom left in FIG. 3, along the notches 34 and 36, is about 1 mm smaller than the maximum width of the fixing element 10 in a longitudinal portion which distally adjoins the two notches 34 and 36. The minimum width of the fixing element 10 at the deepest point of the two notches 34 and 36 is once again about 1 mm smaller than the width of the fixing element 10 at its distal end. The dimensions of a preferred embodiment, specified in millimeters, are specified in the Figures. That also applies for the view at top right in FIG. 3 which is a perspective view of the expansion element 16 and shows the dimensioning of the cross-section thereof. It can be clearly seen from the views at top left and top right in FIG. 3 that the expansion element 16, in a direction transversely with respect to a separating surface between the two sub-elements 12 and 14, is of a cross-sectional dimension which is about 1 mm larger than the cross-sectional dimension of the cavity 18 defined by the two sub-elements 12 and 14, in the corresponding direction.

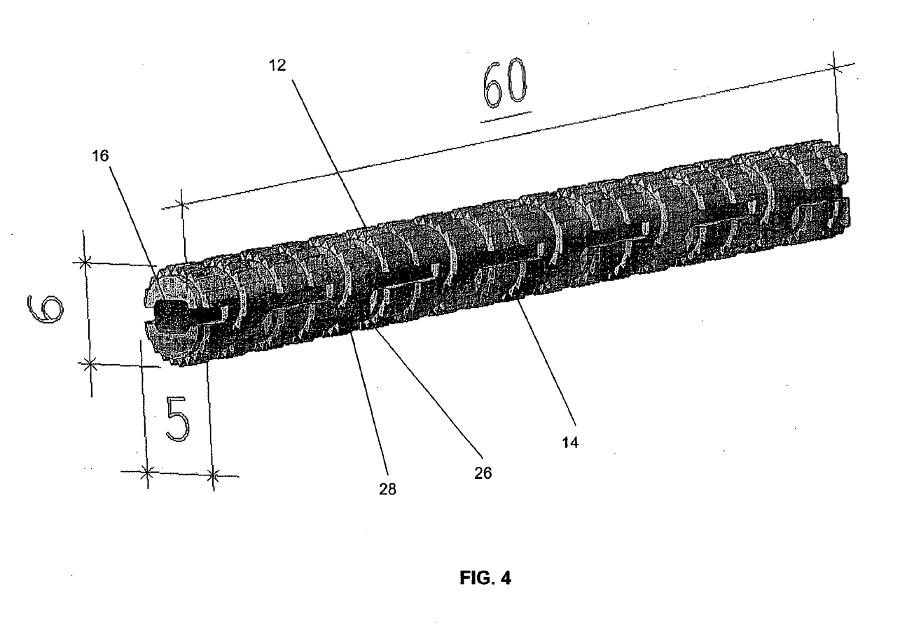

In FIG. 4, the fixing element 10 is shown in its expanded condition in which the expansion element 16 has been pushed into the cavity 18.

The two outer sub-elements 12 and 14 are of such a configuration that in the compact condition of the fixing element 10, as shown in FIG. 2, they impart thereto a cylindrical basic shape in which longitudinal portions 20 of round cross-section and longitudinal portions 22 of stellate cross-section alternate. In the illustrated embodiment, the overall fixing element 10 is of a length of 60 mm and a maximum diameter in the region of the longitudinal portions 22 of stellate cross-section of 5 mm. It will be seen that the diameter of the longitudinal portions 20 of round cross-section is somewhat smaller than the diameter of the longitudinal portions 22 of stellate cross-section. As can also be seen from FIG. 2, the stellate cross-section of the longitudinal portions 22 is derived from prismatic longitudinal ribs on the outside of those longitudinal portions.

The two outer sub-elements 12 and 14 are separated from each other along a separating surface 24 which over large parts thereof extends precisely in the center of the fixing element 10 and is flat. At the level of each third portion 22 of stellate cross-section the separating surface projects in such a way that the one outer sub-element 12 has projections 26 which protrude in the direction of the other sub-element 14 and which engage into corresponding recesses 28 on the other outer sub-element 14. These projections 26 and recesses 28 are so arranged that the two sub-elements 12 and 14 cannot be displaced relative to each other in the longitudinal direction or in the lateral direction, either in the compact condition of the expansion element 10 (see FIG. 2) or in the expanded condition (see FIG. 3) of the expansion element 10.

Depressions 30 and 32 respectively extending along the longitudinal direction of the two outer sub-elements 12 and 14 respectively define the cavity 18 between the two outer sub-elements 12 and 14 into which the expansion element 16 can be inserted. As can be seen from the Figures, essentially over its entire length the cavity 18 is of a uniform rectangular cross-section which is somewhat rounded off at the corners. The cavity 18 narrows somewhat in the region of the distal end of the fixing element 10.

The expansion element 16 is of a cross-section substantially corresponding to the cross-section of the cavity 18. However, the extent of the expansion element 16 in the direction extending transversely relative to the separating surface 24 is somewhat greater than the corresponding extent of the cavity 18 so that the expansion element 16 also actually expands the fixing element 10 when it is pushed into the cavity 18. So that the expansion element 16 can be readily pushed into the cavity 18, even when the fixing element 10 is initially in its compact condition, the corresponding extent of the expansion element 16 continually decreases in the region of the distal end to a dimension which allows the expansion element 16 to be introduced with its distal end leading into the cavity 18 of the fixing element in the compact condition thereof.

The two outer sub-elements 12 and 14 and the expansion element 16 each comprise a bioresorbable thermoplastic material, preferably a polylactide, having the property that after implantation it is gradually absorbed by the human body so that, after a fracture which was fixed by means of the fixing element 10 has healed, there is no longer any implant that has to be removed from the human body. In addition, the material of the two outer sub-elements 12 and 14 and the expansion element 16 has the property that it can be cut with a hot wire so that the fixing element 10 can be of an overlength in relation to a bore in a bone and in that way can be cut off after implantation and insertion of the expansion element 16. A portion of the fixing element 10, that projects externally beyond the bone, can thus be easily cut off. The cutting-off operation is preferably effected with a device having an electrically heated cutting wire with which the thermoplastic material of the fixing element 10 can be easily cut. That advantageously involves a weld between the two outer sub-elements 12 and 14, and the expansion element 16, in the region of the cutting location.

The material of the two outer sub-elements 12 and 14 and optionally also of the expansion element is furthermore preferably provided with a bone growth-stimulating substance which the fixing element 10 after implantation thereof delivers in a dose which is suitable for stimulating bone growth in a manner such as to promote the healing process.

Variants (not shown) of the fixing element according to the invention differ from the fixing element 10 shown in FIGS. 1 through 3 in particular in regard to their length which can be greater than that of the illustrated fixing element 10. Minor deviations in diameter are also possible. Thus it can be up to 1 mm smaller or up to 2 mm larger, than the diameter of the illustrated fixing element. Overall however the fixing element shown in FIGS. 1 through 3 is of a geometry which is advantageously substantially optimized for the intended use, for example for treating femur neck fractures.

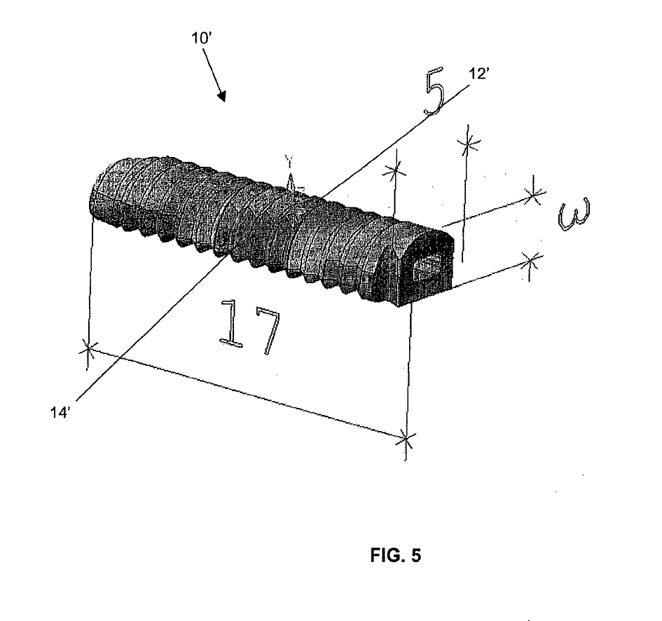

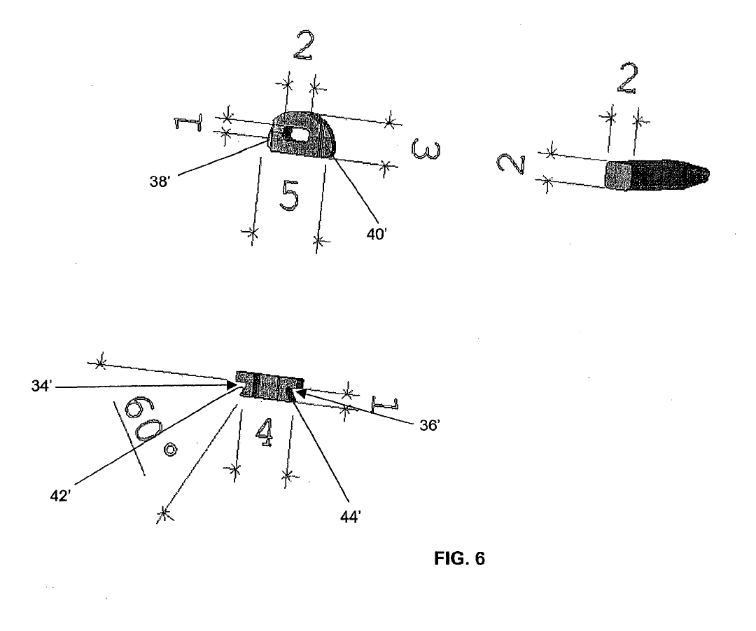

FIGS. 5 through 7 show an alternative fixing element 10′ which, unlike the fixing element shown in FIGS. 1 through 4, is not of a round cross-section, but is flattened on the side of one of the two sub-elements 12′ and 14′. In addition, the fixing element 10′ shown in FIGS. 5 through 7 is shorter than the fixing element of FIGS. 1 through 4.

The fixing element 10′ of FIGS. 5 through 7 is also not optimized for interconnecting bone fragments, but rather for fixing ligaments to a bone.

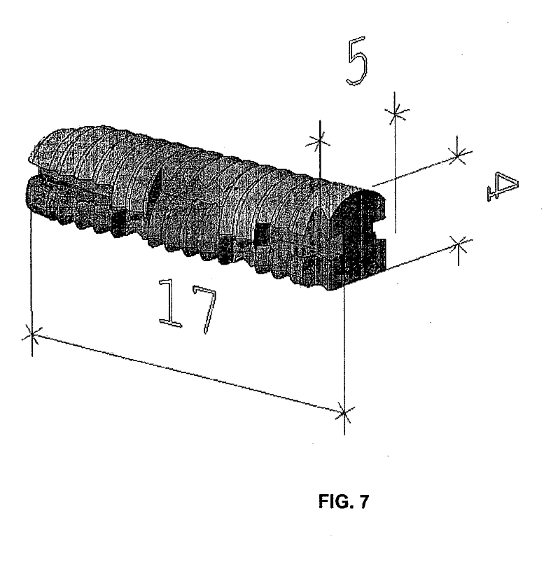

As can be seen from FIGS. 5 and 7 the alternative fixing element 10′, at its proximal end, has a coupling structure formed by two notches 34′ and 36′ and corresponding to that of the fixing element 10 shown in FIGS. 1 through 4.

What is claimed is:

1-15. (canceled)

16. A fixing element for fixing bone fragments, ligaments or the like, comprising at least two outer sub-elements which divide the fixing element in the longitudinal direction with respect to a separating surface between the at least two outer sub-elements, the at least two outer sub-elements are of such a configuration and are so arranged relative to each other that the fixing element is adapted to be transferred from a compact condition into a laterally or radially expanded condition, the fixing element is of a dovetail-like configuration at its proximal end for coupling to an instrument for holding and introducing the fixing element and has two notches which extend on opposite sides of the fixing element transversely with respect to the separating surface between the at least two sub-elements and which respectively extend over the sub-elements and which are so shaped that the depth of the respective notch decreases towards the proximal end of the fixing element and thus the width of the fixing element increases from a viewing direction along the two notches towards the proximal end of the fixing element, the width of the fixing element at the proximal end thereof being smaller than in a longitudinal portion adjoining the notches in the distal direction.

17. A fixing element as set forth in claim 16, wherein the notches are respectively defined by a distal boundary surface extending perpendicularly to the longitudinal axis of the fixing element and a proximal boundary surface extending at an acute angle towards the longitudinal axis of the fixing element.

18. A fixing element as set forth in claim 16, wherein, in a compact condition, the fixing element is of a cylindrical basic shape over the major part of its length.

19. A fixing element as set forth in claim 18, wherein by virtue of the two notches, the width of the fixing element at its proximal end is smaller than the diameter of the cylindrical basic shape by a dimension which is somewhat more than the depth of a notch in the radial direction.

20. A fixing element as set forth in claim 18, wherein the sub-elements provide the fixing element in its compact condition a cylindrical basic shape of a diameter of between 3 and 7 mm and a length of over 60 mm and longitudinal portions of round cross-section and longitudinal portions of stellate cross-section alternate in the longitudinal direction of the fixing element in its compact condition, the longitudinal portions of stellate cross-section are provided with prismatic longitudinal ribs on the outside thereof, wherein the diameter of the longitudinal portions of round cross-section is smaller than the diameter of the longitudinal portions of stellate cross-section.

21. A fixing element as set forth in claim 20, wherein the longitudinal portions of stellate cross-section are of a length of between 1 and 3 mm.

22. A fixing element as set forth in claim 20, wherein the longitudinal portions of round cross-section are of a length of between 0.5 and 1.5 mm.

23. A fixing element as set forth in claim 20, wherein the longitudinal portions of round cross-section are of a cylindrical shape.

24. A fixing element as set forth in claim 18, further including an expansion element of a cross-sectional dimension which over the major part of the length of the expansion element remains the same and is larger than the cross-sectional dimension of the cavity in the corresponding direction.

25. A fixing element as set forth in claim 24, wherein the cavity and the expansion element are each of a rectangular cross-section.

26. A fixing element as set forth in claim 24, wherein, at its distal end, the expansion element is pointed in a wedge shape and narrows in the distal direction to a cross-sectional dimension smaller than the cross-sectional dimension of the cavity in the compact condition of the fixing element.

27. A fixing element as set forth in claim 16, wherein the fixing element comprises bioresorbable plastic material.

28. A fixing element as set forth in claim 27, wherein the bioresorbable plastic material is or contains a polylactide.

29. A fixing element as set forth in claim 27, wherein the bioresorbable plastic material is a thermoplastic material which is adapted to be cut by means of a hot wire.

30. A fixing element as set forth in claim 16, wherein the fixing element is provided with at least one osteosynthesis-stimulating substance.
